(19) United States
(12) Patent Application Publication
(10) Pub. No.: US 2003/0013079 A1
(43) Pub. Date: Jan. 16, 2003

(54) Production of infectious hepadnavirus particles containing foamy retrovirus envelope proteins and methods of using the same

(76) Inventor: Christos J. Petropoulos, Half Moon Bay, CA (US)

Correspondence Address: Cooper & Dunham, LLP, 1185 Avenue of the Americas, New York, NY, 10036, US

(21) Appl. No.: 09/886,711

(22) Filed: Jun. 21, 2001

(51) Int. Cl. C12Q 1/70; C12P 21/06; A61K 39/12; A61K 39/29; C12N 7/00; C12N 7/01; C12N 15/00; C12N 15/09; C12N 15/63; C12N 15/70; C12N 15/74; C12N 5/00; C12N 5/02

(52) U.S. Cl. 435/5; 435/320.1; 424/199.1; 424/225.1; 435/69.1; 435/325; 435/235.1

(57) ABSTRACT

The invention provides a method for producing a hepadnavirus virion that is infectious in vitro which comprises:(a) introducing into a cell (i) a hepadnavirus genome expression vector and (ii) a foamy retrovirus envelope expression vector which comprises a nucleic acid encoding at least a fragment of a foamy virus envelope protein, and (b) culturing the cell thereby producing hepadnavirus virions comprising at least a fragment of a foamy virus envelope protein, wherein the hepadnavirus virions are infectious in vitro. A method for determining susceptibility for an anti-hepadnavirus drug, a method for determining replication capacity of a hepadnavirus from an infected patient, and method for identifying a mutation in a hepadnavirus nucleic acid that confers resistance to an anti-hepadnavirus drug are also provided.

Throughout this application, various publications are referenced by author and date within the text. Full citations for these publications may be found listed alphabetically at the end of the specification immediately preceding the claims. All patents, patent applications and publications cited herein, whether supra or infra, are hereby incorporated by reference in their entirety. The disclosures of these publications in their entireties are hereby incorporated by reference into this application in order to more fully describe the state of the art as known to those skilled therein as of the date of the invention described and claimed herein.

### BACKGROUND OF THE INVENTION

HBV replication

Hepatitis B virus (HBV) particles can be produced by the transient expression of molecular clones of full-length HBV DNA in primary hepatocyte cultures and several hepatoma cell lines. Virus particles produced in this manner resemble the infectious virions (Dane particles) of HBV-infected individuals and their infectivity has been demonstrated in chimpanzees. Unfortunately, HBV particles produced in such in vitro cell systems do not productively infect hepatic cell lines maintained in vitro (e.g. HepG2 cells). This limitation has restricted the study of HBV replication and the development of antiviral drugs. Similarly, the inablility to infect target host cells with HBV particles generated with HBV resistance test vectors is an obstacle in the development of a two cell drug susceptibility assay for HBV as described in U.S. Pat. No. 6,242,187. This block to infection is not understood and may reflect the absence of functional HBV receptors on the surface of available hepatic cell lines, although data supporting other possible explanations have been presented The HBV receptor(s) has yet not been identified.

What is desired, therefore, are means and methods to produce hepadnavirus particles capable of infecting hepatic cell lines maintained in vitro. What is also desired are means and methods to produce hepadnavirus particles which can be used to conduct drug susceptibility and resistance testing, viral fitness assays, and genotypic analysis using a host and target cell, i.e. a two cell in vitro system.

### SUMMARY OF THE INVENTION

Accordingly it is an object of the invention to provide a method for the production of hepadnavirus particles capable of infecting hepatic cell lines maintained in vitro.

A further object of the invention is to provide a method of using infectious hepadnavirus particles to conduct drug susceptibility and resistance testing using a two cell system.

Another object of the invention is to provide a method of using infectious hepadnavirus particles to conduct in vitro drug susceptibility and resistance testing wherein a detectable signal is produced to measure infectivity.

A further object of the invention is to provide in vitro drug susceptibility and resistance testing as described above using the infectious hepadnavirus particles comprising a patient-derived segment.

A further object of the invention is to provide an in vitro method of using infectious hepadnavirus particles to determine replication capacity for patient's hepadnavirus.

Yet another object of the invention is to provide a method of identifying a mutation in a hepadnavirus which confers resistance to a compound which inhibits hepadnavirus replication.

These and other objects may be achieved by the present invention by: producing a hepadnavirus virion that is infectious in vitro which comprises:(a)introducing into a cell (i) a hepadnavirus genome expression vector and (ii) a foamy retrovirus envelope expression vector which comprises a nucleic acid encoding at least a fragment of a foamy virus envelope protein, and (b) culturing the cell thereby producing hepadnavirus virions comprising at least a fragment of a foamy virus envelope protein, wherein the hepadnavirus virions are infectious in vitro.

### BRIEF DESCRIPTION OF THE DRAWINGS

FIG. 1 HBV Indicator Gene Viral Vector

FIG. 2 HBV Resistance Test Vector

FIG. 3 Organization of HBV and HFV Envelope Proteins

### DETAILED DESCRIPTION OF THE INVENTION

This invention provides: a method for producing a hepadnavirus virion that is infectious in vitro which comprises:

(a) introducing into a cell (i) a hepadnavirus genome expression vector and (ii) a foamy retrovirus envelope expression vector which comprises a nucleic acid encoding at least a fragment of a foamy virus envelope protein; and

(b) culturing the cell thereby producing hepadnavirus virions comprising at least a fragment of a foamy virus envelope protein, wherein the hepadnavirus virions are infectious in vitro.

A further embodiment, the invention provides the above method wherein the hepadnavirus genome expression vector lacks a nucleic acid encoding a hepadnavirus envelope protein.

A further embodiment, the invention provides the above method, wherein the hepadnavirus genome expression vector comprises at least one gene from a hepadnavirus genome selected from the group consisting of: a wood chuck hepatitis virus (WHV) genome, a ground squirrel hepatitis (GSHV) virus genome, a duck hepatitis B virus (DHBV) genome, a snow goose hepatitis virus (SGHV) genome, and a human hepatitis B virus (HBV) genome.

A further embodiment, the invention provides the above method, wherein the hepadnavirus genome expression vector comprises a gene from a human hepatitis B virus (HBV) genome.

A further embodiment, the invention provides the above method, wherein the hepadnavirus genome expression vector further comprises an exogenous regulatory element.

A further embodiment, the invention provides the above method, wherein the exogenous regulatory element is a human cytomegalovirus immediate-early gene promoter/enhancer (CMV-IE).

A further embodiment, the invention provides the above method, wherein the foamy retrovirus envelope expression vector comprises at least a fragment of a gene from a foamy virus genome selected from the group consisting of: a siman foamy virus (SFV) genome, a feline foamy virus (FFV) genome, a bovine foamy virus (BFV) genome, a sea lion foamy virus (SLFV) genome, a hampster foamy virus (HaFV) genome, and a human foamy virus (HFV) genome.

A further embodiment, the invention provides the above method, wherein the gene encodes an envelope protein or a fragment thereof.

A further embodiment, the invention provides the above method, wherein the foamy retrovirus envelope expression vector comprises a gene or a fragment of a gene from a human foamy virus (HFV) genome.

A further embodiment, the invention provides the above method, wherein the gene or the fragment of the gene from a human foamy virus (HFV) genome encodes the gp130env envelope gene product or a fragment thereof.

A further embodiment, the invention provides the above method, wherein the cell is a mammalian cell.

A further embodiment, the invention provides the above method, wherein the cell is an avian cell.

A further embodiment, the invention provides the above method, wherein the avian cell avian hepacyte.

A further embodiment, the invention provides the above method, wherein the mammalian cell is a human cell.

A further embodiment, the invention provides the above method, wherein the human cell is a human embryonic kidney cell.

A further embodiment, the invention provides the above method, wherein the mammalian cell is a 293 cell.

A further embodiment, the invention provides the above method, wherein the human cell is a human hepatoma cell.

A further embodiment, the invention provides the above method, wherein the human hepatoma cell is an HepG2 cell or an Huh7 cell.

In another embodiment, the invention provides a hepadnavirus virion that is infectious in vitro which comprises at least a fragment of a foamy retrovirus envelope protein.

In another embodiment, the invention provides a hepadnavirus virion wherein the hepadnavirus virion is isolated.

In another embodiment, the invention provides a hepadnavirus virion wherein the foamy retrovirus is selected from the group consisting of: a siman foamy virus (SFV), a feline foamy virus (FFV), a bovine foamy virus (BFV), a sea lion foamy virus (SLFV), a hampster foamy virus (HaFV), and a human foamy virus (HFV).

In another embodiment, the invention provides a hepadnavirus virion wherein the hepadnavirus virion comprises a chimeric envelope protein which consists essentially of (i) a hepatitis B virus envelope protein domain and (ii) a foamy virus envelope protein domain.

In another embodiment, the invention provides a hepadnavirus virion wherein the hepadnavirus virion further comprises a nucleic acid isolated from a subject infected by a hepadnavirus.

In another embodiment, the invention provides a hepadnavirus virion wherein the nucleic acid isolated from the subject infected by hepadnavirus encodes a reverse transcriptase.

In another embodiment, the invention provides a hepadnavirus virion wherein the hepadnavirus further comprises an indicator nucleic acid.

In another embodiment, the invention provides a cell comprising the hepadnavirus virion.

In another embodiment, the invention provides a cell comprising the hepadnavirus virion, wherein the cell is a mammalian cell.

In another embodiment, the invention provides a cell comprising the hepadnavirus virion, wherein the mammalian cell is a 293 cell.

In another embodiment, the invention provides a cell comprising the hepadnavirus virion, wherein the mammalian cell is a human cell.

In another embodiment, the invention provides a cell comprising the hepadnavirus virion, wherein the human cell is a human kidney cell.

In another embodiment, the invention provides a cell comprising the hepadnavirus virion, wherein the human cell is a human hepatoma cell.

In another embodiment, the invention provides a method for determining susceptibility for an anti-hepadnavirus drug which comprises:

(a) introducing into a first cell:

(i) a hepadnavirus genome expression vector;

(ii) a nucleic acid encoding at least a fragment of a foamy retrovirus envelope protein, and

(iii) an indicator nucleic acid;

(b) culturing the first cell from step (a) so as to produce hepadnavirus virions;

(c) admixing the hepadnavirus virions produced in step (b) with a second cell, wherein the anti-hepadnavirus drug is present with the first cell or the second cell, or with the first and second cell,

(d) measuring the amount of detectable signal produced by the indicator nucleic acid in the second cell, wherein the amount of detectable signal produced is dependent upon hepadnavirus virion infection of the second cell; and

(e) comparing the amount of signal measured in step (d) with the amount signal measured in the absence of the drug, wherein a decrease in the amount of signal measured in the presence of the drug indicates susceptibility to the drug and wherein no change in signal measured or an increase in the amount of signal measured in the presence of the drug indicates resistance to the drug.

In another embodiment, the invention provides the above method for determining susceptibility, wherein the hepadnavirus genome expression vector of step (a) further comprises a nucleic acid derived from a patient infected with hepadnavirus.

In another embodiment, the invention provides the above method for determining susceptibility, wherein the nucleic acid derived from a patient infected with hepadnavirus comprises at least a fragment of a human hepatitis B virus (HBV) gene.

In another embodiment, the invention provides the above method for determining susceptibility, wherein the gene is an HBV P gene, an HCV C gene, an HBV X gene or an HBV S gene.

In another embodiment, the invention provides the above method for determining susceptibility, wherein the nucleic acid derived from a patient infected with hepadnavirus encodes reverse transcriptase.

In another embodiment, the invention provides the above method for determining susceptibility, wherein the second cell is a mammalian cell

In another embodiment, the invention provides the above method for determining susceptibility, wherein the second cell is an avian cell.

In another embodiment, the invention provides the above method for determining susceptibility, wherein the avian cell avian hepacyte.

In another embodiment, the invention provides the above method for determining susceptibility, wherein the mammalian cell is a human cell.

In another embodiment, the invention provides the above method for determining susceptibility, wherein the human cell is a human embryonic kidney cell.

In another embodiment, the invention provides the above method for determining susceptibility, wherein the mammalian cell is a 293 cell.

In another embodiment, the invention provides the above method for determining susceptibility, wherein the human cell is a human hepatoma cell.

In another embodiment, the invention provides the above method for determining susceptibility,, wherein the human hepatoma cell is an HepG2 cell or an Huh7 cell.

In another embodiment, the invention provides the above method for determining susceptibility,, wherein the foamy retrovirus is selected from the group consisting of: a siman foamy virus (SFV), a feline foamy virus (FFV), a bovine foamy virus (BFV), a sea lion foamy virus (SLFV), a hampster foamy virus (HaFV), and a human foamy virus (HFV).

In another embodiment, the invention provides the above method for determining susceptibility, wherein the nucleic acid of step (a) (i) encodes a gp130env envelope protein.

In another embodiment, the invention provides the above method for determining susceptibility, wherein the nucleic acid of step (a) (i) encodes a chimeric envelope protein which consists essentially of (i) a hepatitis B virus envelope protein domain and (ii) a foamy virus envelope protein domain.

In another embodiment, the invention provides the above method for determining susceptibility, wherein the second cell expresses on its surface a protein which binds human foamy virus envelope protein.

In a further embodiment the invention provides a method for determining replication capacity of a hepadnavirus from an infected patient comprising:

(a) introducing into a first cell:

(i) a hepadnavirus genome expression vector;

(ii) a nucleic acid encoding at least a fragment of a foamy retrovirus envelope protein, and

(iii) an indicator nucleic acid;

(b) culturing the cell from (a) so as to produce hepadnavirus virions;

(c) admixing the hepadnavirus virions produced in step (b) with a second cell,

(d) measuring the amount of detectable signal produced by the indicator nucleic acid in the second cell, wherein the amount of detectable signal produced is dependent upon hepadnavirus virion infection of the second cell;

(e) normalizing the measurement of step (d); and

(f) comparing the normalized measurement of step (e) with the amount signal measured when steps (a) through (d) are carried out with a control reference hepadnavirus, wherein an increase in signal compared to the control indicates an increased replication capacity and a decrease in signal measured compared to the control indicates a decreased replication capacity of the hepadnavirus from the infected patient.

In a further embodiment the invention provides a method for determining susceptibility for an anti-hepadnavirus drug which comprises:

(a) introducing into a cell:

(i) a hepadnavirus genome expression vector;

(ii) a nucleic acid encoding at least a fragment of a foamy retrovirus envelope protein, and

(iii) an indicator nucleic acid;

(b) culturing the cell from step (a);

(c) contacting the cell with the anti-hepadnavirus drug;

(d) measuring the amount of detectable signal produced by the indicator nucleic acid in the cell; and

(e) comparing the amount of signal measured in step (d) with the amount signal measured in the absence of the drug, wherein a decrease in the amount of signal measured in the presence of the drug indicates susceptibility to the drug and wherein no change in signal measured or an increase in the amount of signal measured in the presence of the drug indicates resistance to the drug.

In a further embodiment the invention provides the above method, wherein the hepadnavirus genome expression vector of step (a) further comprises a nucleic acid derived from a patient infected with hepadnavirus.

In a further embodiment the invention provides the above method, wherein the nucleic acid derived from a patient infected with hepadnavirus comprises at least a fragment of a human hepatitis B virus (HBV) gene.

In a further embodiment the invention provides the above method, wherein the gene is an HBV P gene or an HBV C gene.

In a further embodiment the invention provides the above method, method for identifying a mutation in a hepadnavirus nucleic acid that confers resistance to an anti-hepadnavirus drug which comprises:

(a) sequencing the hepadnavirus nucleic acid prior to use of the anti-hepadnavirus drug;

(b) measuring susceptibility of the hepadnavirus sequenced in step (a) to the drug according to the method of claim 50;

(c) exposing the hepadnavirus to the drug so as to produce a decrease in the susceptibility of the hepadnavirus to the drug measured in step (b);

(d) comparing the sequence determined in step (a) with the sequence of the hepadnavirus following the exposure to the drug of step (c) so as to identify a mutation in the hepadnavirus nucleic acid that confers resistance to the anti-hepadnavirus drug.

In a further embodiment the invention provides the above method, wherein measuring step (b) comprises measuring susceptibility of the hepadnavirus sequenced in step (a) to the anti-hepadnavirus drug using a two cell assay.

In a preferred embodiment of the invention, the invention provides a method for the production of infectious Human Hepatitis B Virus (HBV) particles by pseudotyping HBV virions using envelope proteins derived from the Human Foamy Virus (HFV).

In yet another embodiment of the invention, a method is provided for the production of infectious HBV particles by pseudotyping using chimeric envelope proteins derived from specific functional domains of the HBV and HFV envelope proteins.

Further embodiments of the invention include the production of other various hepadnaviruses, using human foamy virus envelope proteins or chimeric envelope proteins derived from specific functional domains of hepadnavirus and human foamy virus envelope proteins. Examples of other hepadnaviruses include, but are not restricted to, woodchuck hepatitis virus (WHV), ground squirrel hepatitis (GSHV), duck hepatitis B virus (DHBV), snow goose hepatitis virus (SGHV), and other less-well documented hepadnaviruses isolated from cats, rodents, marsupials and birds.

Other embodiments of the invention include the production of hepadnaviruses using various other foamy virus envelope proteins or chimeric envelope proteins derived from specific functional domains of hepadnavirus and various other foamy virus envelope proteins. Examples of other foamy viruses (also referred to as spumaviruses) include, but are not restricted to, simian foamy virus (SFV), feline foamy virus (FFV), bovine foamy virus (BFV), sea lion foamy virus (SLFV), and hamster foamy virus (HaFV).

Other embodiments of the invention include the production of HBV or other various hepadnaviruses using retrovirus envelope proteins or chimeric envelope proteins derived from specific functional domains of hepadnavirus and retrovirus virus envelope proteins. Examples of other retroviruses include, but are not restricted to:

(i) Type B retroviruses (mouse mammary tumor virus);

(ii) Mammalian C-type retroviruses (ecotropic murine leukemia virus, amphotropic murine leukemia virus, gibbon ape leukemia virus, feline leukemia virus, subgroup B); (iii) Avian sarcoma/leukosis retroviruses (subgroups A, B/E, D);

(iv) Type D retroviruses (Mason-Pfizer monkey virus, simian retrovirus 1 and 2);

(v) Human T cell leukemi viruses (type I and II) and bovine leukemia virus;

(vi) Lentiviruses (human immunodeficiency virus type 1 and 2, equine infectious anemia virus, maedi/visna virus);

(vii) Fish retroviruses (walleye pike leukemia and sarcoma viruses, snakehead fish retrovirus);

(viii) Drosophila retrovirus (gypsy).

Other embodiments of the invention include the production of hepadnaviruses using envelope proteins derived from other various enveloped viruses or chimeric envelope proteins derived from specific functional domains of the envelope proteins of hepadnaviruses and other various enveloped viruses. Examples of other enveloped enveloped viruses include, but are not restricted to, togaviruses, flaviviruses, coronaviruses, rhabdoviruses, filoviruses, paramyxoviruses, orthoviruses, bunyaviruses, arenaviruses, herpesviruses, poxviruses, iridovirusesand rotaviruses.

In another embodiment, the invention provides a method for measuring the replication of HBV, and the replication of various other hepadnaviruses.

In another embodiment, the invention provides a method for measuring the susceptibility of HBV and other hepadnaviruses to drugs that inhibit HBV reverse transcriptase, and the reverse transcriptases of other hepadnaviruses.

In another embodiment, the invention provides a method for identifying new and/or additional inhibitors of HBV reverse transcriptase, and the reverse transcriptases of other hepadnaviruses.

The means and methods for measuring HBV replication of the present invention can be applied to the identification of novel inhibitors of HBV replication including, but not limited to, cccDNA formation, virion assembly, and egress from the cell.

In another embodiment, the invention provides a method for identifying mutations in the HBV P gene that alter the susceptibility of HBV to reverse transcriptase inhibitors.

The means and methods of the present invention for identifying mutations that alter susceptibility to reverse transcriptase inhibitors can be adapted to other steps in HBV replication, including, but not limited to, cccDNA formation, virion assembly and egress from the cell.

In another embodiment, the invention provides a method for identifying mutations in the HBV P gene that alter the replicative capacity, or “fitness” of HBV.

The means and methods of the present invention for identifying HBV P gene mutations that alter replicative capacity can be applied to the identification of mutations in other HBV genes (core (C), surface (S), and transactivation (X)) that alter HBV replicative capacity.

In another embodiment, the invention provides a method for using measurements of HBV drug susceptibility to guide the antiviral treatment of individuals infected with HBV.

In another embodiment, the invention provides a method for using replicative capacity measurements to guide the treatment of individuals failing anti-HBV drug treatment.

The embodiments of the present invention are achieved by using envelope proteins derived from a foamy retrovirus to to produce pseudotyped hepadnavirus virions.

Foamy virus (Spumavirus) replication

The replication pathways of hepadnaviruses (which includes HBV) and retroviruses are similar in that both package a genomic length RNA and utilize reverse transcriptase (RT) to generate a double stranded (ds) DNA that serves as the template for transcription of viral genes in infected cells. Foamy viruses (also referred to as spumaviruses) comprise an atypical genus within the retrovirus group in that several aspects of their replication pathway are distinct from that of all other retrovirus genera. Notably, these unusual aspects of the foamy virus replication closely resemble features of hepdnavirus replication, including HBV, and could reflect a common evolutionary link between hepadnaviruses and foamy viruses. Foamy viruses have been reported to infect a variety of cell types from a variety of mammalian and avian species, suggesting that foamy virus receptors represent ubiquitously expressed cell surface proteins.

Similarities Between Hepadnavirus and Foamy Virus Replication

Both hepadnaviruses and retroviruses utilize RT during replication. During hepadnavirus replication, the conversion of a packaged single stranded pre-genomic RNA transcript to double stranded genomic DNA by RT takes place before virus particles enter new host cells. Conversely, during retrovirus replication, this step occurs after the virus entry step. Recent studies indicate that unlike all other known retroviruses, an estimated 10-15% of foamy virus particles contain genomic length double stranded DNA (Yu et al., (1996), “Human Foamy Virus Replication-a Pathway Distinct from That of Retroviruses and Hepadnaviruses”, Science 271: 1579-1582; Yu et al., (1999), “Evidence That the Human Foamy Virus Genome is DNA”, J. Virol. 70: 1250-1254). In this group of retroviruses, significant amounts of reverse transcription occurs before virus particles infect new cells, thus resembling the RT step in hepadnavirus replication.

In newly infected cells, both hepadnaviruses and retroviruses produce large amounts of viral core protein. For hepadnaviruses this is the C protein and for retroviruses it includes the Gag polyprotein consisting of domains that comprise the matrix (MA), capsid (CA) and nucleocapsid (NC) proteins. In hepadnaviruses and foamy viruses the core proteins transiently localize within the nucleus. The de novo synthesized core proteins (Gag polyprotein) of all other known retroviruses are restricted to the cytoplasm of infected cells. The NC domain of all retrovirus Gag polyproteins, except the foamy viruses, contains a highly conserved cysteine-histidine (Cys-His) motif that plays an essential role in the binding of NC to retrovirus genomic RNA and packaging. Berkowitz, R. et al. 1996. RNA packaging. Curr. Top. Microbiol. Immunol. 5 214:177-218. The NC domain of foamy virus Gag polyproteins lack the Cys-His motif, but contains several regions rich in glycine and arginine (Gly-Arg). Schliephake, A. W., et al. 1994. Nuclear localzation of foamy virus Gag precursor protein. J. Virol. 68:4946-4954. Yu, S. F., et al. 1996. The carboxyl terminus of the human foamy virus Gag protein contains separable nucleic acid binding and nuclear transport domains. J. Virol. 70:8255-8262. One of these regions was shown to function as a nuclear localization signal. Analogous Gly-Arg motifs exist in the hepadnavirus core (C proteins and are likely to play important roles in RNA packaging and nuclear localization of the C protein Hatton, T., et al. 1992. RNA- and DNA-binding activities in hepatitis B virus capsid protein: a model for their roles in virus replication. J. Virol. 66:5232-5241. Nassal, M. 1992, The arginine-rich domain of the hepatitis B virus core protein is required for pergenome encapsidation and productive viral positive- strand DNA synthesis but not for virus assembly. J. Virol. 66:4107-4116.

All of the known retroviruses, except the foamy viruses, express their pol genes (RT and integrase (IN) proteins) as Gag-Pol polyproteins. Jacks, T. 1990. Translational suppression in gene expression in retroviruses and retrotransposons, p. 93-124. In R. Wanstrom and P. K. Vogt (ed.), Retroviruses: strategies of replication. Springer-Verlag, Berlin, Germany. In contrast, foamy viruses express their Pol polyproteins separately from Gag polyproteins, resembling Pol expression in the hepadnaviruses Yu, S. F., et al., 1996. Human foamy virus replication—a pathway distinct from that of retroviruses and hepadnaviruses. Science 271:1579-1582. Lochelt, M., et al. 1991. Construction of an infectious DNA clone of the full-length human spumaretrovirus genome and mutagenesis of the bel 1 gene. Virology 184:43-54., Yu, S. F., et al. 1996. Productive persistent infection of hematopoietic cells by human foamy virus. J. Virol. 70:1250-1254.

Retrovirus particle formation occurs exclusively within the cytoplasm, but may vary in precise location depending on the specific virus. All known retroviruses, except the foamy viruses, bud from the cell surface and thus acquire their outer envelope membrane from the plasma membrane. In contrast, both foamy viruses and hepadnaviruses bud from the endoplasmic reticulum (ER) and thus acquire their envelope membrane from the intracellular membrane compartment. The latter may explain why both the hepadnaviruses and the spumaviruses are largely cell associated, while other retroviruses are easily shed from the cell Zemba, M., et al. 1998. The carboxy-terminal p3^{Gag} domain of the human foamy virus Gag precursor is required for efficient virus infectivity. Virology 247:7-13. Yu, S. F., et al., 1993. Analysis of the role of the bel and bet open reading frames of human foamy virus by using a new quantitative assay. J. Virol. 67:6618-6624.

Prior to, and during virion formation both hepadnaviruses and retroviruses concentrate specific envelope proteins within a specific host cell membrane compartment that serves as the source of virus envelope membrane. In the case of hepadnaviruses these are the three surface proteins encoded by the S gene (large, middle and small S) and for retroviruses they are the surface (SU) and transmembrane (TM) proteins encoded by the envelope (env) gene. Both hepadnavirus S proteins and foamy virus TM proteins are reported to contain sorting motifs that localize these proteins within the ER membrane compartment Goepfer, P. A., et al. 1997. A sorting motif localizes the foamy virus glycoprotein to the endoplasmic reticulum. J. Virol. 71:778-784, T. Kamimura et al., and P. Roingeard, 1990. For many, if not all known retroviruses, excluding the foamy viruses, env protein expression is dispensible for the egress of virions from the cell (albeit env deficient particles are not infectious). In contrast, the egress of infectious foamy virus particles from the cell is dependent on env gene expression (4 and 22). Similarly, the assembly of infectious hepadnavirus virions is dependent on the expression of S gene products, and more specifically budding requires appropriate expression of the large S protein. Bruss & Ganum 1991 from table.

The features shared by foamy viruses and hepadnaviruses are summarized in Table 1.

In the case of the human hepatitis B virus, HBV particles produced by transient transfection of cultured cells are infectious in vivo, but not in vitro. The block to infection may be due to the absence of an appropriate HBV receptor on the cell surface. In contrast, human foamy virus (HFV) has a very broad host range and is capable of infecting a wide variety of cell lines. This suggests that the HFV receptor may be a ubiquitously expressed cell surface protein.

HBV and HFV replication pathways have several similar features with respect to virion assembly and budding. The invention describes the means and methods to exploit similarities between the replication pathways of a hepadnavirus, such as HBV and a foamy retrovirus, such as HFV in order to circumvent obstacles that restrict hepadnavirus infection in cell culture systems. In a preferred embodiment, HFV envelope proteins, or chimeric envelope proteins containing specific functional domains of the HBV and HFV envelope proteins, can be used to generate HBV particles that are capable of using the human foamy virus receptor to enter a wide variety of cell types.

As used herein, “hepadnavirus genome expression vector” refers to a vector(s) that comprises at least a fragment of a of hepadnavirus genome and is capable of transient transcription of the hepadnavirus RNA and hepadnavirus protein production following introduction into an appropriate cell line.

An “foamy retrovirus envelope expression vector” refers to a vector that comprises at least a fragment of a foamy retrovirus envelope gene and is capable of transiently producing a foamy retrovirus envelope protein following introduction into an appropriate cell line. An “indicator nucleic acid” refers to a nucleic acid that either directly or through a reaction gives rise to a measurable or noticeable aspect or detectable signal, e.g. a color or light of a measurable wavelength or in the case of DNA or RNA used as an indicator a change or generation of a specific DNA or RNA structure. Preferred examples of an indicator gene is the E. coli lacz gene which encodes beta-galactosidase, the luc gene which encodes luciferase either from, for example, Photonis pyralis (the firefly) or Renilla reniformis (the sea pansy) , the E. coli phoA gene which encodes alkaline phosphatase, green fluorescent protein and the bacterial CAT gene which encodes chloramphenicol acetyltransferase. Additional preferred examples of an indicator gene are secreted proteins or cell surface proteins that are readily measured by assay, such as radioimmunoassay (RIA), or fluorescent activated cell sorting (FACS), including, for example, growth factors, cytokines and cell surface antigens (e.g. growth hormone, I1-2 or CD4, respectively). “Indicator gene” is understood to also include a selection gene, also referred to as a selectable marker. Examples of suitable selectable markers for mammalian cells are dihydrofolate reductase (DHFR), thymidine kinase, hygromycin, neomycin, zeocin or E. coli gpt. In the case of the foregoing examples of indicator genes, the indicator gene and the patient-derived segment are discrete, i.e. distinct and separate genes. In some cases a patient-derived segment may also be used as an indicator gene. In one such embodiment in which the patient-derived segment corresponds to more than one viral gene which is the target of an anti-viral, one of said viral genes may also serve as the indicator gene. The indicator nucleic acid or indicator gene may be “functional” or “non-functional” as described in U.S. Pat. No. 6,242,187.

A “hepadnavirus indicator vector” or “indicator gene viral vector” refers to a DNA vector that contains elements of the hepadnavirus genome and an indicator gene, such as firefly luciferase and is capable of transient transcription of an RNA. The RNA contains the signals/elements required for packaging of the RNA into hepadnavirus virions and for reverse transcription of the RNA transcript by the hepadnavirus polymerase and for the expression of the indicator gene,

A “packaging host cell” or “first cell” refers to a cell that can support transient expression of the hepadnavirus genomic and foamy retrovirus envelope expression vectors.

A “target cell” or “second cell” refers to cells that express a foamy retrovirus envelope receptor and are capable of supporting hepadnavirus replication once foamy retrovirus pseudotyped hepadnavirus virions have entered the cell via the foamy retrovirus receptor. What is meant by “foamy retrovirus pseudotyped hepadnavirus virions” are hepadnavirus virions containing one or more proteins derived from a foamy retrovirus.

As used herein, “patient-derived segment” encompasses nucleic acid segments derived from human and various animal species. Such species include, but are not limited to chimpanzees, horses, cattles, cats and dogs.

Patient-derived segments can also be incorporated into be described vectors, such as the hepadnavirus expression vector using any of several alternative cloning techniques. For example, cloning via the introduction of class II restriction sites into both the plasmid backbone and the patient-derived segments or by uracil DNA glycosylase primer cloning or a method of recombination or seamless cloning.

The patient-derived segment may be obtained by any method of molecular cloning or gene amplification, or modifications thereof, by introducing patient sequence acceptor sites, as described below, at the ends of the patient-derived segment to be introduced into the described vectors, such as the hepadnavirus expression vector. For example, in a gene amplification method such as PCR, restriction sites corresponding to the patient-sequence acceptor sites can be incorporated at the ends of the primers used in the PCR reaction. Similarly, in a molecular cloning method such as cDNA cloning, said restriction sites can be incorporated at the ends of the primers used for first or second strand cDNA synthesis, or in a method such as primer-repair of DNA, whether cloned or uncloned DNA, said restriction sites can be incorporated into the primers used for the repair reaction. The patient sequence acceptor sites may also be regions designed to permit homologous recombination or complementary annealing between the patient derived segment and the hepadnavirus expression vector. The patient sequence acceptor sites and primers are designed to improve the representation of patient-derived segments. Sets of vectors having designed patient sequence acceptor sites provide representation of patient-derived segments that would be underrepresented in one vector alone.

As used herein, “replication capacity” is defined herein is a measure of how well the virus replicates. This may also be referred to as viral fitness. In one embodiment, replication capacity can be measured by evaluating the ability of the virus to replicate in a single round of replication.

As used herein, “control resistance test vector” is defined as a resistance test vector comprising a standard hepadnavirus sequence (for example, HBVayw and an indicator gene.

As used herein, “normalizing” is defined as standardizing the amount of the expression of indicator gene measured relative to the number of viral particles giving rise to the expression of the indicator gene. For example, normalization is measured by dividing the amount of luciferase activity measured by the number of viral particles measured at the time of infection. “Plasmids” and “vectors” are designated by a lower case p followed by letters and/or numbers. The starting plasmids herein are either commercially available, publicly available on an unrestricted basis, or can be constructed from available plasmids in accord with published procedures. In addition, equivalent plasmids to those described are known in the art and will be apparent to the ordinarily skilled artisan.

Construction of the vectors of the invention employs standard ligation and restriction techniques which are well understood in the art (see Ausubel et al., (1987) Current Protocols in Molecular Biology, Wiley—Interscience or Maniatis et al., (1992) in Molecular Cloning: A laboratory Manual, Cold Spring Harbor Laboratory, N.Y.). Isolated plasmids, DNA sequences, or synthesized oligonucleotides are cleaved, tailored, and religated in the form desired. The sequences of all DNA constructs incorporating synthetic DNA can be confirmed by DNA sequence analysis (Sanger et al. (1977) Proc. Natl. Acad. Sci. 74, 5463-5467).

The development of a method to generate infectious pseudotyped hepadnavirus virions having envelope proteins derived from a foamy retrovirus enables the development of in vitro cell based assays for hepadnaviruses, including but not limited to drug susceptibility and resistance essays, viral fitness assays, and genotypic assays to identify hepadnavirus mutations which confer drug resistance.

The following examples are presented to further illustrate and explain the invention and should not be taken as limiting in any regard.

### EXAMPLE 1

Pseudotyping Hepatitis B Virus Using Envelope Proteins Derived from Human Foamy Virus

This example provides a means and methods for generating HBV virions that are capable of infecting primary cell cultures and established cell lines that express the receptor for Human Foamy Virus (HFV) . The means and methods provided herein describe the procedures for incorporating HFV envelope proteins into the membrane of HBV and the infection of target cells that are permissive for HFV infection, i.e. express HFV receptors on the cell surface. HBV virions produced by the method enter the cell by binding and interacting with the HFV receptor, thereby circumventing the normal HBV entry pathway, which is thought to involve the HBV surface protein (S) and an, as yet, unidentified host cell HBV receptor. It is widely held that the inability of HBV to infect cultured cells is likely to be due to a block(s) at the attachment and/or entry steps. The means and methods for producing infectious HBV by pseudotyping with HFV envelope proteins provided in this example can be adapted to other hepadnaviruses, some of which may serve as useful animal models for HBV disease, for example duck and woodchuck hepadnaviruses. Addition, the means and methods for producing infectious HBV by pseudotyping with HFV envelope proteins can be adapted to pseudotyping HBV and other hepadnaviruses with the envelope proteins of other foamy viruses (spumaviruses), retroviruses, and a variety of enveloped viruses.

The system for the production of HBV particles pseudotyped with HFV envelope proteins and the successful infection of cultured cells involves the following components;

(i) HBV genome expression vector: a DNA vector that comprises the HBV genome and is capable of transient transcription of HBV RNA and HBV protein production following introduction into an appropriate cell line.

(ii) HBV indicator vector: a DNA vector that contains elements of the HBV genome and an indicator gene, such as firefly luciferase and is capable of transient transcription of an RNA. The RNA contains the signals/elements required for packaging of the RNA into HBV virions and for reverse transcription of the RNA transcript by the HBV polymerase and for the expression of the indicator gene,

(iii) HFV envelope expression vector: a DNA vector that comprises the HFV envelope gene and is capable of transiently producing HFV envelope proteins following introduction into an appropriate cell line.

(iv) Packaging host cell or first cell: cells that can support transient expression of HBV genomic and HFV envelope expression vectors.

(v) Target cell or second cell: cells that express the HFV envelope receptor and are capable of supporting HBV replication once HFV pseudotyped HBV virions have entered the cell via the HFV receptor.

HBV genome expression vectors are capable of producing HBV particles following their introduction into packaging host cells. HBV gene expression can be regulated by HBV regulatory elements, or by exogenous regulatory elements derived from other sources, e.g. the human cytomegalovirus immediate-early gene promoter/enhancer (CMV-IE). In a preferred embodiment of this invention, expression of the HBV genome is regulated by the CMV-IE promoter/enhancer. HBV genome expression vectors may also contain an indicator gene, such as firefly luciferase. In this case, the vectors are referred to as “HBV indicator gene viral vectors” or more generally as “indicator gene viral vectors”. The indicator gene provides a sensitive and convenient mechanism for measuring the infectivity of target cells following infection by virus produced in host packaging cells. The amount of indicator gene product, i.e. luciferase activity, produced in target cells is a direct measure of a single round of HBV replication. HBV indicator gene viral vectors can be used to assemble HBV “Resistance/fitness test vectors” by replacing specific HBV sequences of the HBV indicator gene viral vector with HBV gene sequences (e.g. P gene reverse transcriptase sequences) derived from a variety of other sources. Sources may include patient samples harboring drug sensitive or drug resistant strains of HBV (e.g. viruses sensitive or resistant to lamivudine, [3TC]), and molecular clones of HBV that possess defined RT sequences that contain or lack drug resistance associated mutations (M550V).

The HFV envelope expression vector contains the HFV envelope gene region and is used to produce the HFV envelope gene product (gp130env). The gp130env is a polyprotein that is cleaved in the cytoplasmic membrane compartment by a cellular “furin-like” protease to produce the mature envelope surface (gp8OSU) and transmembrane (gp48TM) . Together, SU and TM function in host cell recognition and entry of HFV. The introduction of HFV envelope expression vectors along with HBV genome vectors into host packaging cells results in the production of HBV virions bearing HFV envelope proteins in the viral membrane (pseudotyped virus particles). Expression of HFV envelope in host packaging cells can be regulated by a variety of regulatory elements including, but not limited to the CMV-IE promoter/enhancer, or the HFV promoter/enhancer. In a preferred embodiment of this invention, the HFV envelope expression vector is assembled by inserting the HFV envelope gene region into an expression vector that contains the CMV-IE promoter/enhancer (e.g. pCXAS, Petropoulos et al., 1999 Cite Full Ref).

Packaging host cells may include a wide variety of human or mammalian cell lines including, but not limited to, human embryonic kidney cells (HEK293) and human hepatoma cells (HepG2, Huh7). The ideal packaging host cell transiently produces large numbers of HFV pseudotyped HBV virions following the introduction of HBV genome expression vector and HFV envelope expression vector DNAs.

Target cells may include primary cells and cell lines, and more specifically primary hepatocytes and cell lines of hepatic origin, including but not limited to HepG2 cells and Huh7 cells (ref). The ideal target cell expresses HFV receptor(s) on the cell surface and supports HBV replication steps that are downstream of virus attachment and entry.

To produce infectious HBV virus particles an HBV genome expression vector plus an HFV envelope expression vector is introduced into host packaging cells. Several days later, HFV pseudotyped HBV particles produced by the host packaging cells are harvested and used to inoculate target cells. Several days after inoculation, the infectivity of target cells is measured. The introduction of HBV genome expression vector and HFV envelope expression vector DNAs into host packaging cells can be performed by a variety of well-established procedures including, but not limited to calcium-phosphate-DNA precipitation and electroporation. Measuring the infectivity of target cells by HBV can be performed by a variety of well-established procedures including, but not limited to the detection of HBV antigens (e.g. antibody based Western blot or ELISA detection), or HBV nucleic acids (e.g. PCR, RT-PCR, Northern blot, Southern blot detection).

In a preferred embodiment of this invention, the HBV genome expression vector and the HFV envelope expression vector are regulated by the CMV-IE promoter/enhancer. The HBV genome contains a luciferase indicator gene. The host packaging cell is HEK293. The HBV genome expression vector and the HFV envelope expression vector are introduced into host packaging cells by calcium-phosphate-DNA precipitation. Five to ten micrograms of each vector DNA preparation are used. After transfection, host packaging are incubated for 24-72 hours. Cells plus culture media are collected and frozen and thawed to release cell-associated virions. The media is centrifuged and filtered and the filtrate serves as the stock of HFV pseudotyped HBV for infection of host target cells. The target host cell is HepG2 or Huh7. Infected cells are lysed 48-72 hours after infection and luciferase activity is measured in the cell lysate. The amount of luciferase activity detected in infected cells serves as a direct measure of a single round of HBV replication.

### EXAMPLE 2

Pseudotyping Hepatitis B Virus Using Chimeric Envelope Proteins Derived from Human Foamy Virus and Hepatitis B Virus

This example provides a means and methods for generating HBV virions that are capable of infecting primary cell cultures and established cell lines that express the receptor for Human Foamy Virus (HFV). The means and methods provided herein describe the procedures for incorporating HBV/HFV chimeric envelope proteins into the membrane of HBV and the infection of target cells that are permissive for HFV infection, i.e. express HFV receptors on the cell surface. HBV virions produced by the method enter the cell by binding and interacting with the HFV receptor, thereby circumventing the normal HBV entry pathway, which is thought to involve the HBV surface protein (S) and an, as yet, unidentified host cell HBV receptor. It is widely held that the inability of HBV to infect cultured cells is likely to be due to a block(s) at the attachment and/or entry steps. Based on this example, it is obvious that the means and methods for producing infectious HBV by pseudotyping with HBV/HFV chimeric envelope proteins can be adapted to other hepadnaviruses, some of which may serve as useful animal models for HBV disease, for example duck and woodchuck hepadnaviruses. Based on this example, it is also obvious that the means and methods for producing infectious HBV by pseudotyping with HBV/HFV chimeric envelope proteins can be adapted to pseudotyping HBV and other hepadnaviruses with chimeric envelope proteins derived from other foamy viruses (spumaviruses), retroviruses, and a variety of enveloped viruses.

The system for the production of HBV particles pseudotyped with HBV/HFV chimeric envelope proteins and the successful infection of cultured cells may involve the following components;

HBV genome expression vector: a DNA vector that contains the HBV genome and is capable of transient transcription of HBV RNA and HBV protein production following introduction into an appropriate cell line,

HBV indicator gene viral vector: a DNA vector that contains elements of the HBV genome and an indicator gene, such as firefly luciferase and is capable of transient transcription of an RNA. The RNA contains the signals/elements required for packaging of the RNA into HBV virions and for reverse transcription of the RNA transcript by the HBV polymerase and for the expression of the indicator gene,

HBV/HFV chimeric envelope expression vector: a DNA vector that contains the sequences coding for a HBV/HFV chimeric envelope gene and is capable of transiently producing the HBV/HFV chimeric envelope proteins following introduction into an appropriate cell line,

Packaging host cells: cells that can support transient expression of HBV genomic and HFV envelope expression vectors,

Target host cells: cells that express the HFV envelope receptor and are capable of supporting HBV replication once HBV particles pseudotyped with the HBV/HFV chimeric envelope have entered the cell via the HFV receptor.

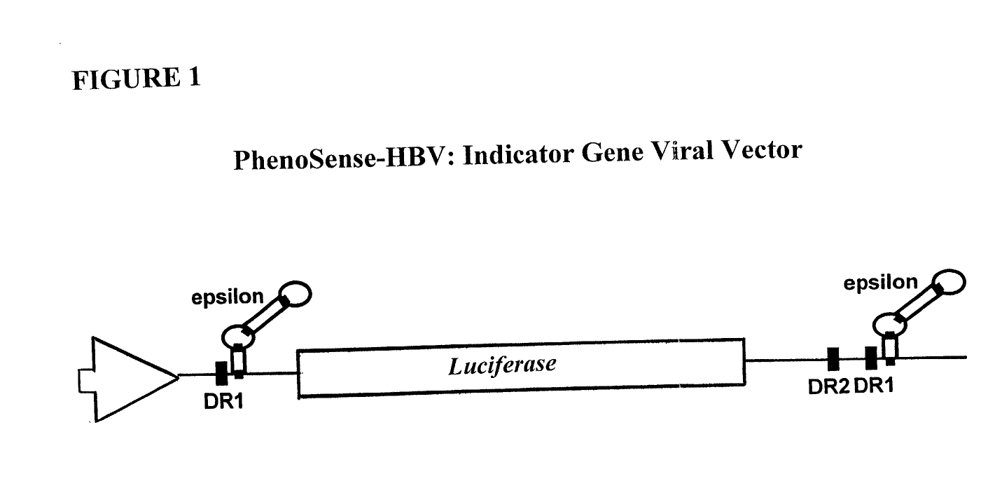

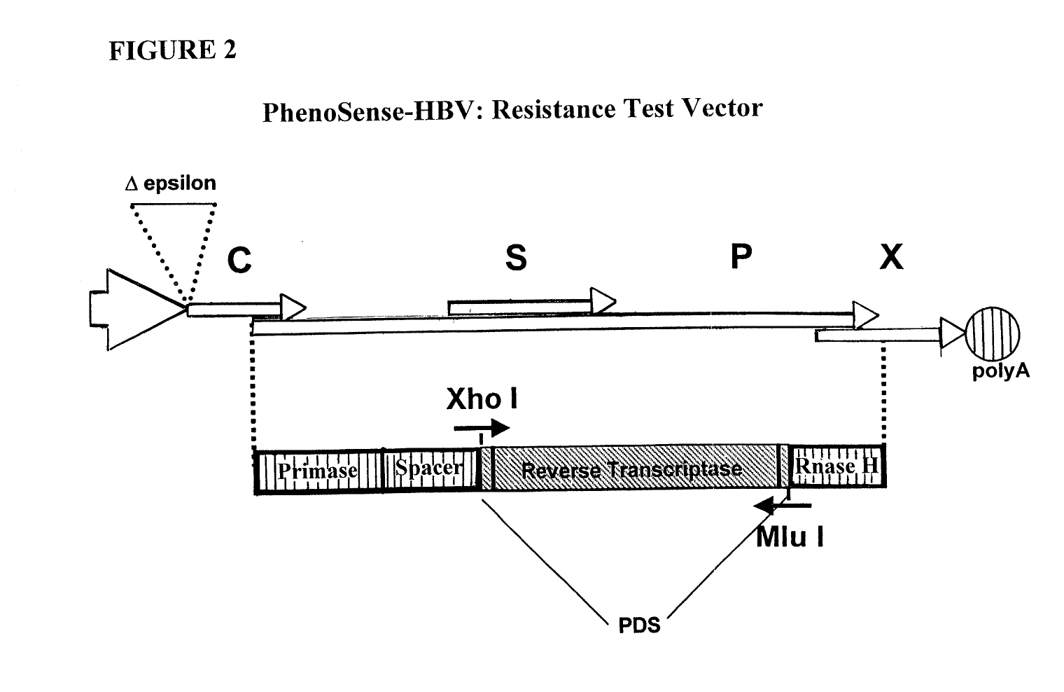

HBV genome expression vectors are capable of producing HBV particles following their introduction into packaging host cells. HBV gene expression can be regulated by HBV regulatory elements, or by exogenous regulatory elements derived from other sources, e.g. the human cytomegalovirus immediate-early gene promoter/enhancer (CMV-IE). In a preferred embodiment of this invention, expression of the HBV genome is regulated by the CMV-IE promoter/enhancer. HBV genome expression vectors may also contain an indicator gene, such as firefly luciferase. In this case, the vectors are referred to as “HBV indicator gene viral vectors” (FIG. 1). The indicator gene provides a sensitive and convenient mechanism for measuring the infectivity of host target cells following infection by virus produced in host packaging cells. The amount of indicator gene product, i.e. luciferase activity, produced in host target cells is a direct measure of a single round of HBV replication. HBV genome expression vectors and/or HBV indicator gene viral vectors can be used to assemble “HBV Resistance/fitness test vectors” (see FIG. 2 and Example 3 below). HBV Resistance/fitness test vectors are produced by replacing specific HBV sequences of the HBV genome expression vector or the HBV indicator gene viral vector with HBV gene sequences (e.g. P gene reverse transcriptase sequences) derived from a variety of other sources. Sources may include patient samples harboring drug sensitive or drug resistant strains of HBV (e.g. viruses sensitive or resistant to lamivudine, [3TC]), and molecular clones of HBV that possess defined RT sequences that contain or lack drug resistance associated mutations (M550V).

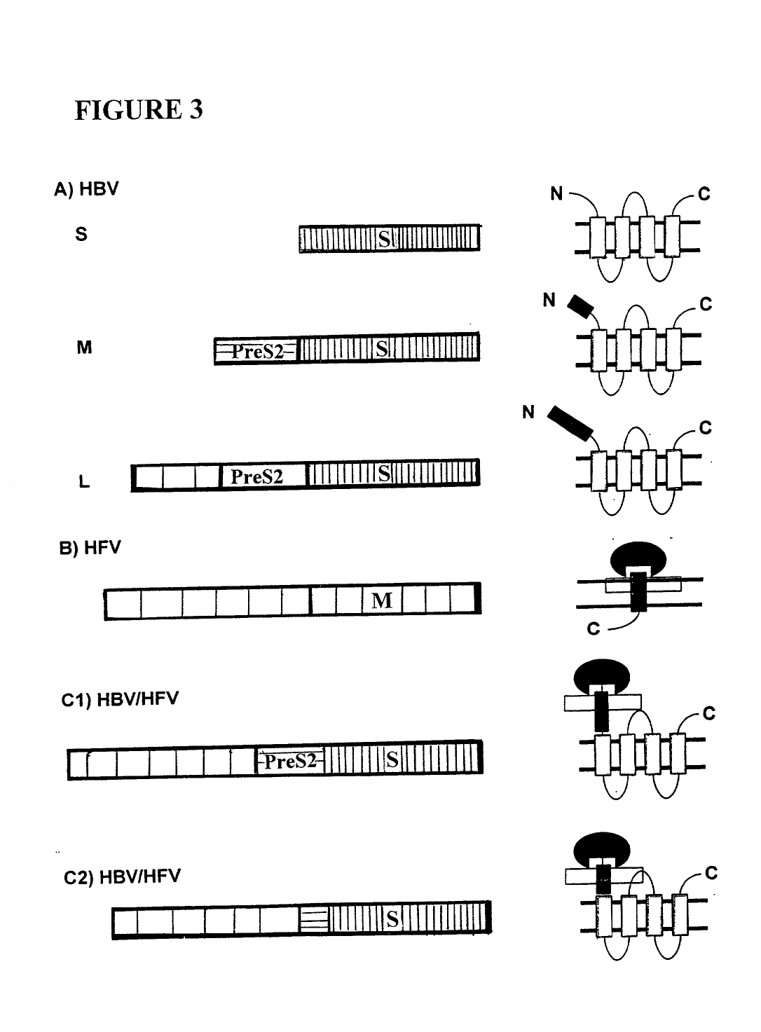

The HFV gp130env envelope is a polyprotein that is cleaved in the cytoplasmic membrane compartment by a cellular “furin-like” protease to produce the mature envelope surface (gp8OSU) and transmembrane (gp48TM). Together, SU and TM function in host cell recognition and entry of HFV. The HBV PreS1/PreS2/S gene codes for three different proteins depending on the promoter used. The three proteins S, M and L contain identical C-terminii and differ in the presence or absence of the PreS1and/or PreS2 domains (See FIG. 3). The HBV/HFV chimeric envelope expression vector contains sequences that encode a chimeric protein which contains amino acids derived from the entire S domain and additional PreS1 and PreS2 sequences of the HBV virus covalently linked to amino acids of the HFV SU (gp80) envelope gene region. In a preferred embodiment of this invention the HBV/HFV chimeric envelope contains the HFV SU region fused in frame to the entire HBV S and PreS2 and N-terminal deleted PreS1 sequences. In another 5 preferred embodiment of this invention the HBV/HFV chimeric envelope contains the HFV SU region fused in frame to the entire HBV S and N-terminal deleted PreS1 and C-terminal deleted PreS2 sequences. The HBV/HFV chimeric envelope expression vector is used to produce the HBV/HFV chimeric envelope gene product. The introduction of HBV/HFV chimeric envelope expression vectors along with HBV genome vectors into host packaging cells results in the a production of HBV virions bearing HBV/HFV chimeric envelope proteins in the viral membrane (pseudotyped virus particles). Expression of HBV/HFV chimeric envelope in host packaging cells can be regulated by a variety of regulatory elements including, but not limited to the CMV-IE promoter/enhancer, or the HFV promoter/enhancer or the HBV S promoter. In a preferred embodiment of this invention, the HBV/HFV chimeric envelope expression vector is assembled by inserting the HBV/HFV chimeric envelope gene sequences into an expression vector that contains the CMV-IE promoter/enhancer (e.g. pCXAS, Petropoulos et al., 1999).

Packaging host cells may include a wide variety of human or mammalian cell lines including, but not limited to, human embryonic kidney cells (HEK293) and human hepatoma cells (HepG2, Huh7). The ideal packaging host cell transiently produces large numbers of HBV virions pseudotyped with the HBV/HFV chimeric envelope protein following the introduction of HBV genome expression vector and HBV/HFV chimeric envelope expression vector DNAs.

Target host cells may include primary cells and cell lines, and more specifically primary hepatocytes and cell lines of hepatic origin, including but not limited to HepG2 cells and Huh7 cells. The ideal target host cell expresses HFV receptor(s) on the cell surface and supports HBV replication steps that are downstream of virus attachment and entry.

To produce infectious HBV virus particles an HBV genome expression vector plus an HBV/HFV chimeric envelope expression vector is introduced into host packaging cells. Several days later, HBV particles pseudotyped with the HBV/HFV chimeric envelope produced by the host packaging cells are harvested and used to inoculate target host cells. Several days after inoculation, the infectivity of target cells is measured. The introduction of HBV genome expression vector and HBV/HFV chimeric envelope expression vector DNAs into host packaging cells can be performed by a variety of well-established procedures including, but not limited to calcium-phosphate-DNA precipitation and electroporation. Measuring the infectivity of target cells by HBV can be performed by a variety of well-established procedures including, but not limited to the detection of HBV antigens (e.g. antibody based Western blot or ELISA detection), or HBV nucleic acids (e.g. PCR, RT-PCR, Northern blot, Southern blot detection).

In a preferred embodiment of this invention, the HBV genome expression vector and the HFV envelope expression vector are regulated by the CMV-IE promoter/enhancer. The HBV genome contains a luciferase indicator gene. The host packaging cell is HEK293. The HBV genome expression vector and the HBV/HFV chimeric envelope expression vector are introduced into host packaging cells by calcium-phosphate-DNA precipitation. Five to ten micrograms of each vector DNA preparation are used. After transfection, host packaging are incubated for 24-72 hours. Cells plus culture media are collected and frozen and thawed to release cell-associated virions. The media is centrifuged and filtered and the filtrate serves as the stock of HBV particles pseudotyped with the HBV/HFV chimeric envelope for infection of host target cells. The target host cell is HepG2 or Huh7. Infected cells are lysed 48-72 hours after infection and luciferase activity is measured in the cell lysate. The amount of luciferase activity detected in infected cells serves as a direct measure of a single round of HBV replication.

### EXAMPLE 3

Methods for measuring HBV drug susceptibility and replication capacity (“viral fitness”)

This example provides the means and methods for accurately and reproducibly measuring HBV drug susceptibility and identifying new/additional inhibitors or HBV replication. This example further provides the means and methods for measuring the replicative capacity of HBV that exhibits reduced susceptibility to reverse transcriptase inhibitors, or drugs/compounds that target other steps in HBV replication. The means and methods for measuring drug susceptibility and replicative capacity can be adapted to other hepadnaviruses, some of which may serve as useful animal models for HBV disease, for example duck and woodchuck hepadnaviruses.

Drug susceptibility and replicative capacity testing are carried out using the means and methods described in U.S. Pat. No. 6,242,187 and U.S. Ser. No. 09/766,344, the contents of which are hereby incorporated herein by reference. HBV drug susceptibility and replication capacity testing are performed using “HBV Resistance/Fitness test vectors”, “HFV envelope packaging vectors”, “packaging host cells” and “target cells” as described.

Packaging host cells may include a wide variety of human or mammalian cell lines including, but not limited to human embryonic kidney cells (HEK293) and human hepatoma cells (HepG2, Huh7) . The ideal packaging host cell will produce large numbers of pseudotyped HBV virions following the introduction of an HBV “Resistance/Fitness test vector” DNA. Target host cells may include primary cells and cell lines, and more specifically primary hepatocytes and cell lines of hepatic origin, including but not limited to HepG2 cells and Huh7 cells. The ideal target host cell will express HFV receptor(s) on the cell surface and support HBV replication steps that are downstream of virus attachment and entry.

HBV Resistance/Fitness test vectors express HBV genes and are capable of producing HBV particles following their introduction into packaging host cells. HBV Resistance/Fitness test vectors also contain a functional indicator gene, such as firefly luciferase. The amount of luciferase activity produced in target cells following infection is a direct measure of HBV replication. HBV Resistance/fitness test vectors are constructed with HBV P gene sequences (encoding reverse transcriptase activity) derived from a variety of sources. Sources may include patients samples harboring drug sensitive or drug resistant strains of HBV (e.g. lamivudine), and molecular clones of HBV that possess defined RT sequences that contain or lack drug resistance associated mutations (M550V).

To produce infectious HBV virus particles, packaging host cells, such as HEK293, are co-transfected with HBV Resistance/Fitness test vector DNA plus HFV envelope packaging vector DNA described above in Example 1. The envelope packaging vector must be capable of producing HFV envelope proteins; gp8OSU, gp48TM (for example PCXAS-HFVenv), or chimeric envelope proteins containing specific functional domains of HBV and HFV envelope proteins (pCXAS-HBV/HFVenv). The HFV pseudotyped HBV particles viral that are produced by the host packaging cells are harvested several days after transfection and used to infect target host cell (cell freeze/thaw may increase titer by releasing cell-associated virions). Several days after infection, target cells are lysed and luciferase activity is measured.

The amount of luciferase activity detected in the infected cells is used as a direct measure of “infectivity”, also referred to as “replicative capacity” or “in vitro fitness”, i.e. the ability of the virus to complete a single round of replication. Relative fitness is assessed by comparing the amount of luciferase activity produced by a test virus (e.g. RT sequences derived from a patient sample) to the amount of luciferase activity produced by a well-characterized reference virus derived from a molecular clone of HBV, HBVayw. Viruses that are “less fit” than the reference virus will produce less luciferase after infection of target cells. Viruses that are “more fit” than the reference virus will produce more luciferase after infection of target cells. Fitness measurements are expressed as a percent of the reference virus, for example 25%, 50%, 75%, 100% or 125% of reference.

Susceptibility to antiviral drugs (e.g. reverse transcriptase inhibitors) is assessed by comparing the amount of luciferase activity produced by a test virus (e.g. RT sequences derived from a patient sample) in the presence of drug to the amount of luciferase activity produced by the same test virus in the absence of drug. Viruses are tested over a broad range of drug concentrations in order to generate inhibition curves that enable accurate quantitation of drug activity (Petropoulos et al., 1999. Typically, drug activity is represented as the concentration of drug required to inhibit 50%, or 95% of virus replication, referred to as IC50 and IC95, respectively. Replication of test viruses that are susceptible to a drug will be inhibited by the same concentration of the drug as a well-characterized drug sensitive reference virus HBVayw. In this case, the IC50 of the test virus will be essentially the same as the IC50 of the reference virus. Replication of test viruses that exhibit decreased susceptibility to a drug will be inhibited at a higher drug concentration than a well-characterized drug sensitive reference virus. In this case, the IC50 of the test virus will be higher than the IC50 of the reference virus. Replication of test viruses that exhibit increased susceptibility to a drug will be inhibited at a lower drug concentration than a well-characterized drug sensitive reference virus. In this case, the IC50 of the test virus will be lower than the reference virus.

### EXAMPLE 4

Methods for Identifying Genetic Mutations Associated with Changes in HBV Drug Susceptibility And/or Replicative Capacity.

This example provides a means and method for identifying mutations in reverse transcriptase that alter HBV drug susceptibility and/or replication fitness. The means and methods for identifying mutations that alter HBV drug susceptibility and/or replication fitness can be adapted to other steps in the HBV replication cycle, including, but not limited to cccDNA formation, virus assembly, and virus egress. This example also provides a means and method for quantifying the affect that specific reverse trascriptase mutations have on drug susceptibility and/or replicative capacity. A means and method for quantifying the affect that specfic reverse transcriptase mutations have on drug susceptibility and/or replicative capacity can be adapted to mutations in other viral genes involved in HBV replication, including the C and X genes.

HBV Resistance/fitness test vectors are constructed as described and referenced in Example 1. Resistance/fitness test vectors derived from patient samples or clones derived from the resistance/fitness test vector pools, or resistance/fitness test vectors engineered by site directed mutagenesis to contain specific mutations, are tested in drug susceptibility and fitness assays to determine accurately and quantitatively the drug susceptibility and relative fitness compared to a well-characterized reference standard. In another embodiment of the invention, the drug susceptibility and/or fitness of the patient virus is compared to viruses collected from the same patient at different time points, for example prior to initiating therapy, before or after changes in drug treatment, or before or after changes in virologic (RNA copy number), immunologic (CD4 T-cells), or clinical (opportunistic infection) indicators of disease progression. The results of patient samples can be further examined for changes in reverse transcriptase activity associated with the observed changes in drug susceptibility and/or relative fitness.

Reverse transcriptase activity of patient HBV samples

Reverse transcriptase activity can be measured by any number of widely used assay procedures, including but not limited to homopolymeric extension (e.g. oligo dT:poly rC) using conventional or real time PCR based on molecular beacons (reference Kramer?) or 5′exonuclease activity (Lie and Petropoulos, 1996). In one embodiment, virion associated reverse transcriptase activity is measured using a quantitative PCR assay that detects the 5′exonuclease activity associated with thermo-stable DNA polymerases. In one embodiment of the invention, the HBV RT activity of the patient virus is compared to the HBV RT activity of a reference virus (i.e. “wildtype”) that has not been exposed to reverse transcriptase inhibitors or other antiviral drugs. In another embodiment, the HBV RT activity is compared the HBV RT activity of viruses collected from the same patient at different time points, (for example prior to initiating therapy, before or after changes in drug treatment, or before or after changes in virologic (RNA copy number), immunologic (CD4 T-cells), or clinical (opportunistic infection) indicators of disease progression.

Genotypic Analysis of Patient HBV Samples

Resistance/fitness test vector DNAs, either pools or individual clones which make up the pools, are analyzed by any number of widely practiced genotyping methods (e.g. nucleic acid sequencing, differential probe hybridization, oligonucleotide array hybridization). In one embodiment of the invention, patient HBV sample sequences are determined using viral RNA purification, RT/PCR and dideoxynucleotide chain terminator sequencing. The sequence that is determined is compared to reference sequences present in the database, or is compared to a sample from the patient prior to initiation of therapy, if available. The genotype is examined for sequences that are different from the reference or pre-treatment sequence and correlated to the observed change in drug susceptibility and/or replicative capacity.

Drug Susceptibility and Replicative Fitness Analysis of Site Directed Mutants

Genotypic changes that are observed to correlate with changes in HBV drug susceptibility and/or replicative fitness are evaluated, by constructing resistance/fitness test vectors containing the specific mutation on a defined genetic background derived from a well-characterized, drug susceptible virus (i.e. “wildtype”). Mutations may be incorporated alone and/or in combination with other mutations that are thought to modulate the drug susceptibility and/or fitness of a virus. Mutations are introduced into the resistance/fitness test vectors through any of the widely known methods for site-directed mutagenesis. In one embodiment of this invention the mega-primer PCR method for site-directed mutagenesis is used. Resistance/fitness test vectors containing a specific mutation, or group of mutations, is tested using the drug susceptibility and/or fitness assays described in Example 3. The fitness of the mutant virus is compared to that of the reference virus lacking the specific mutation(s). Observed changes in drug susceptibility and/or fitness are attributed to the specific mutations introduced into the resistance test vector. In a related embodiment of the invention, resistance/fitness test vectors containing site directed mutations in reverse transcriptase that result in amino acid substitutions at position 550 (M550V, M550I) are constructed and tested for drug susceptibility and/or fitness. The fitness results enable the correlation between specific reverse transcriptase amino acid substituions and changes in drug susceptibility and/or fitness.

### REFERENCES

1. Achong, B. G., W. A. Mansell, M. A. Epstein, and P. Clifford. 1971. An unusual virus in cultures from a human nasopharyngeal carcinoma. J. Natl. Cancer Inst. 46:299-307.

2. Ali, M., G. P. Taylor, R. J. Pitman, D. Parker, A. Rethwilm, R. Cheingsong-popov, J. N. Weber, P. D. Bieniasz, J. Bradley, and M. O. McClure. 1996. No evidence of antibody to human foamy virus in widespread human populations. AIDS Res. Hum. Retroviruses 12:1473-1483.

3. Allan, J. S., S. R. Broussard, M. G. Michaels, T. E. Starzt, K. L Leighton, E. M. Whitehead, A. G. Comuzzie, R. E. Lanford, M. M. Leland, W. M. Switzer, and W. Hieneine. 1998. Amplification of simian retroviral sequences from human recipients of baboon liver transplants. AIDS Res. Hum. Retroviruses 14:821-824.

4. Baldwin, D. N., and M. L. Linial. 1998. The roles of Pol and Env in the assembly pathway of human foamy virus. J. Virol. 72:3658-3665.

5. Baunacli, G., B. Maurer, H. Hahn, M. Kranz, and A. Rethwilm. 1993. Functional analysis of human foamy virus accessory reading frames. J. Virol. 67:5411-5418.

6. Berkowitz, R., J. Fisher, and S. P. Goff. 1996. RNA packaging. Curr. Top. Microbiol. Immunol. 214:177-218.

7. Bieniasz, P. D., A. Rethwilm, R. Pitman, M. D. Daniel, I. Chrystie, and M. O. McClure. 1995. A comparative study of higher primate foamy viruses, including a new virus from a gorilla. Virology 207:217-228.

8. Blair W. S. H. Bogerd, and B. R. Cullen. 1994. Genetic analysis indicates that the human foamy virus Bel-1 protein contains a transcription activation domain of the acidic class. J. Virol. 68:3803-3808.

9. Bock, M. M. Heinkelein, D. Lindemann, and A. Rethwilm. 1998. Cells expressing the human foamy virus (HFV) accessory Bet protein are resistant to productive HFV superinfection. Virology 250:194-204.

10. Bodem, J., M. Lochelt, I. Winkler, R. P. Flower, H. Delius, and R. M. Flugel. 1996. Characterization of the spliced pol transcript of feline foamy virus—the splice acceptor site of the pol transcript is located in gag of foamy viruses. J. Virol. 70:9024-9027.

11. Bodem, J., M. Lochelt, P. Yang, and R. M. Flugel. 1997. Regulation of gene expression by human foamy virus and potentials of foamy viral vectors. Stem Cells 15:141-147.

12. Bour, S., U. Schubert, K. Peden, and K. Strebel. 1996. The envelope glyco-protein of human immunodeficiency virus type 2 enhances viral particle release: a Vpu-like factor? J. Virol. 70:820-829.

13. Broussard, S. R., A. G. Comuzzie, K. L Leighton, M. M. Leland, E. M. Whitehead, and J. S. Allan. 1997. Characterization of new simian foamy viruses from African nonhuman primates. Virology 237:349-359.

14. Campbell, M, C. Eng, and P. A. Luciw. 1996. The simian foamy virus type 1 transcriptional transactivator (Tas) binds and activates an enhancer element in the gag gene. J. Virol. 70:6847-6855.

15. Campbell, M., L. Renshaw-Gegg, R. Renne, and P. A. Luciw. 1994. Characterization. of the internal promoter of simian foamy viruses. J. Virol. 68: 4811-4820.

16. Coffin, J. M., S. H. Hughes, and H. E. Varmus. 1997. Retroviruses. Cold Spring Harbor Press, Plainview, N.Y.

16a. Comstock, K., C. Meiering, and M. Linial. Unpublished data.

17. Cordonnier, A., J. F. Casella, and T. Heidmann. 1995. Isolation of novel human endogenous retrovirus-like elements with foamy virus-related pol sequence. J. Virol. 69:5890-5897.

17a. Eastman, S., and M. Linial. Unpublished data.

18. Enders, J., and T. Peebles. 1954. Propagation in tissue culture of cytopathogenic agents from patients with measles. Proc. Soc. Biol. Med. 86:277-287.

19. Enssle, J., N. Fischer, A. Moebes, B. Mauer, U. Smola, and A. Rethwilm. 1997. Carboxy-terminal cleavage of the human foamy virus gag precursor molecule is an essential step in the viral life cycle. J. Vitol. 71:7312-7317.

20. Enasse, J., I. Jordan, B. Mauer, and A. Rethwilm. 1996. Foamy virus reverse transcriptase is expressed independently from the Gag protein. Proc. Natl. Acad. Sci. USA 93:4137-4141.

21. Erlwein, O., P. D. Bieniasz, and M. O. McClure. 1998. Sequences in pol are required for transfer of human foamy virus-based vectors. J. Virol. 72.5510-5516.

22. Fischer, N., M. Heinkelein, D. Lindemann, J. Enssle, C. Baum, E. Werder, H. Zentgraf, J. G. Muller, and A. Rethwilm. 1998. Foamy virus particle formation. J. Virol. 72:1610-1615.

23. Flugel, R. M. 1992. Spumaviruses: a group of complex retroviruses. J. Acquired Immune Defic. Syndr. 4:739-759.

24. Flugel, R. M., A. Rethwilm, B. Maurer, and G. Darai. 1987. Nucleotide sequence analysis of the env gene and its flanking regions of the human spumaretrovirus reveals two novel genes. EMBO J. 6:2077-2084.

25. Ganem, D. 1996. Hepadnaviridae: the viruses and their replication, p. 2703-2737. In B. N. Fields, D. M. Knipe, P. M. Howley, R. M. Chanock, and J. L. Melnick (ed.), Fields Virology. Lippincott-Raven, Philadelphia, Pa.

26. Giron, M. L., H. de The, and A. Saib. 1998. An evolutionarily conserved splice generates a secreted Env-Bet fusion protein during human foamy virus infection. J. Virol. 72:4906-4910.

27. Giron, M. L, S. Colas, J. Wybier, F. Rozain, and R. Emanoil-Ravier. 1997 Expression and maturation of human foamy virus gag precursor polypeptides. J. Virol. 71:1635-1639.

27a. Goepfert, P., and M. Mulligan. Personal communication.

28. Goepfert, P. A., K. L. Shaw, G. D. Ritter, and M. J. Mulligan. 1997. A sorting motif localizs the foamy virus glycoprotein to the endoplasmic reticulum. J. Virol. 71:778-784.

29. Hahn, H., G. Baunach, S. Brautigam, A. Mergia, D. Neumann-Haefelin, M. D. Daniel, M. O. McClure, and A. Rethwilm. 1994. Reactivity of primate sera to foamy virus Gag and Bet proteins. J. Gen. Virol. 75:2635-2644.

30. Hatton, T., S. Zhou, and D. N. Standring, 1992. RNA- and DNA-binding activities in hepatitis B virus capsid protein: a model for their roles in virus replication. J. Virol. 66:5232-5241.

31. He, F., W. S. Blair, J. Fukushima, and B. R. Cullen. 1996. The human foamy virus Bel-1 transcription factor is a sequence-specific DNA binding protein. J. Virol. 70:3902-3908.

32. Heinkelein, M., M. Schmidt, N. Fischer, A. Moebes, D. Lindemann, J. Enssle, and A. Rethwilm. 1998. Characterization of a cis-acting sequence in the pol region required to transfer human foamy virus vectors. J. Virol. 72:6307-6314.

33. Herchenroder, O., R. Renne, D. Loncar, E. K, Cobb, K. K. Murthy, J. Schneider, A. Mergia, and P. A. Luciw. 1994. Isolation, cloning, and sequencing of simuan foamy viruses from chimpanzees (SFVcpz):high homology to human foamy virus (HFV). Virology 201:187-199.

34. Herchenroder, O., R. Turek, D. Neumann-Haefelin, A. Rethwilm, and J. Schneider, 1995. Infectious proviral clones of chimpanzee foamy virus (SFVcpz) generated by long PCR reveal close functional relatedness to human foamy virus. Virology 214:685-689.

35. Herniou, E., J. Martin, K. Miller, J. Cook, M. Wilkinson, and M. Tristem. 1998 Retroviral diversity and distribution in vertebrates. J. Virol. 72:5955-5966.

36. Holzschu, D. L., M. A. Delaney, R. W. Renshaw, and J. W. Casey. 1998. The nucleotide sequence and spliced pol mRNA levels of the nonprimate spumavirus bovine foamy virus. J. Virol. 72:2177-2182.

37. Hooks, J. J., and C. J. Gibbs. 1975. The foamy viruses. Bacteriol. Rev. 39:169-185.

38. Hruska, J. F., and K. K. Takemoto. 1975. Biochemical properties of a hamster syncytium-forming (“foamy”) virus. J. Natl. Cancer Inst. 54:601-605.

39. Jacks, T. 1990. Translational suppression in gene expression in retroviruses and retrotransposons, p. 93-124. In R. Swanstrom and P. K. Vogt (ed.), Retroviruses: strategies of replication. Springer-Verlag, Berlin, Germany.

40. Johnson, R. H., A. A. Oginnusi, and P. W. Ladds. 1983. Isolations and serology of bovine spumavirus. Aust. Vet. J. 60:147.

41. Jordan, I., J. Enssle, E. Guttler, B. Mauer, and A. Rethwilm. 1996. Expression of human foamy virus reverse transcriptase involves a spliced pol mRNA. Virology 224:314-319.

42. Kang, Y. B., W. S. Blair, and B. R. Cullen. 1998 Identification and functional characterizaation of a high-affinity Bel-1 DNA binding site located in the human foamy virus internal promoter. J. Virol. 72:504-511.

43. Kennedy-Stoskopf, S., M. K. Stoskopf, M. A. Eckhaus, and J. D. Strandberg. 1986. Isolation of a retrovirus and a herpesvirus from a captive California sea lion. J. Wild. Dis. 22:156-164.

44. Kertayadnya, I. G., R. H. Johnson, I. Abher, and G. W. Burgess. 1988. Detection of immunological tolerance to bovine spumavirus (BSV) with evidence for salivary excretion and spread of BSV from the tolerant animal. Vet. Microbiol. 16:35-39.

45. Kogel, D., M. Aboud, and R. M. Flugal. 1995. Molecular biological characterization of the human foamy virus reverse transcriptase and ribonuclease H domains. Virology 213:97-108.

46. Konvalinka, J., M. Lochelt, H. Zentgraf, R. M. Flugel, and H. G. Krausslich. 1995. Active spumavirus proteinase is essential for virus infectivity but not for formation of the Pol polyprotein. J. Virol. 69:7264-7268.

47. Kupiec, J. J., J. Tobaly-Tapiero, M. Canivet, M. Santillana-Hayat, R. M. Flugel, J. Peries, and R. Emaneil-Ravier. 1988. Evidence for a gapped linear duplex DNA intermediate in the replicative cycle of human and simian spumaviruses. Nucleic Acids Res. 16:9557-9565.

48. Lee, A. H., H. Y. Lee, and Y. C. Sung, 1994. The gene expression of human foamy virus does not require a post-transcriptional transactivator. Virology 204:409-413.

49. Lindemann, D., M. Bock, M. Schweizer, and A. Rethwilm. 1997. Efficient pseudotyping of murine leukemia virus particles with chimeric human foamy virus envelope proteins. J. Virol. 71:4815-4820.

49a. Lindemann, D., and A. Rethwilm. 1998. Characterization of a human foamy virus 170-kilodalton Env-Bet fusion protein generated by alternative splicing J. Virol. 72:4088-4094.

50. Lochelt, M., and R. M. Flugel. 1996. The human foamy virus pol gene is expressed as a Pro-Pol polyprotein and not as a Gag-Pol fusion protein. J. Virol. 70:1033-1040.

51. Lochelt, M., W. Muranyi, and R. M. Flugel. 1993. Human foamy virus genome possesses an internal, bel-1 dependent and functional promoter Proc. Natl. Acad. Sci. USA 90:7317-7321.

52. Lochelt, M., S. F. Yu, M. L. Linial, and R. M. Flugel. 1995. The human foamy virus internal promoter is required for efficient gene expression and infectivity. Virology 206:601-610.

53. Lochelt, M., H. Zentgraf, and R. M. Flugel. 1991. Construction of an infectious DNA clone of the full-length human spumaretrovirus genome and mutagenesis of the bel 1 gene. Virology 184:4354.

54. Lutz, H. 1990. Feline retroviruses: a brief review. Vet. Microbiol. 23:131-146.

55. Maurer, B., H. Bannert, G . Darai, and R. M. Flugel. 1988. Analysis of the primary structure of the long terminal repeat and the gag and pol genes of the human spumaretrovirus. J. Virol. 62:1590-1597.

56. Mergia, A. 1994. Simian foamy virus type 1 contains a second promoter located at the 3′end of the env gene. Virology 199:219-222.

57. Mochizuid, M., M. Akuzawa, and H. Nagatoma. 1990. Serological survey of the Iriomote cat (Felis iriomotensis) in Japan. J. Wild. Dis. 26:236-245.

58. Moebes, A., J. Enssle, P. D. Bieniasz, M. Heinkelein, D. Lindemann, D. Bock, M. O. McClure, and A. Rethwilm. 1997. Human foamy virus reverse transcription that occurs late in the viral replication cycle. J. Virol. 71:7305-7311.

59. Morozov, V. A., T. D. Copeland, K. Nagashima, M. A. Gonda, and S. Oroszlan. 1997. Protein composition and morphology of human foamy virus intracellular cores and extracellular particles. Virology 228:307-317.

60. Muranyi, W., and R. M. Flugel. 1991. Analysis of splicing patterns of human spumaretrovirus by polymerase chain reaction reveals complex RNA structures. J. Virol. 65:727-735.

61. Nassal, M. 1992. The arginine-rich domain of the hepatitis B virus core protein is required for pregenome ericapsidation and productive viral positive-strand DNA synthesis but not for virus assembly. J. Virol. 66:4107-4116.

62. Nestler, U., M. Heinkelein, M. Lucke, J. Meixensberger, W. Scheurlen, A. Kretschmer, and A. Rethwilm. 1997. Foamy virus vectors for suicide gene therapy. Gene Ther. 4:1270-1277.

63. Netter, K. O., A. Rethwilm, B. Maurer, arid V. ter Meulen. 1990. Identification of the major immunogenic structural proteins of human foamy virus. J. Gen. Virol. 71:1237-1241.

64. Neumann-Haefelin, D., U. Fleps, R. Renne, and M. Schweizer. 1993. Foamy viruses. Intervirology 35:196-207.

65. Neumann-Haefelin, D., M. Schweizer, B. Corsten, and B. Matz. 1986. Detection and characterization of infectious DNA intermediates of a primary foamy virus. J. Gen. Virol. 67:1993-1999.

66. Pahl, A. and R. M. Flugel. 1995. Characterization of the human spumaretrovirus integrase by site-directed mutagenesis, by complementation analysis, and by swapping the zinc finger domain of HIV-1, J. Biol. Chem. 270:2957-2966.

67. Pfrepper, K. I., H. R Rackwitz, M. Schesizer, H. Held, M. Lochelt, and R. M. Flugel. 1998. Molecular characterization of proteolytic processing of the Pol proteins of human foamy virus reveals novel features of the viral protease. J. Virol. 72:7648-7652.

68. Pfrepper, K. I., M. Lochelt, M. Schnolzer, and R. M. Fluget. 1997. Expression and molecular characterization of an enzymatically active recombinant human spumaretrovirus protease. Biochem. Biophys. Res. Common. 237: 548-553.

69. Pietschmann, T., M. Heinkelein, M. Heldmann, H. Zentgraf, A. Rethwilm, and D. Lindemann. Foamy virus capsids require the cognate envelope proteins for particle export. Submitted for publication.

70. Rethwilm, A., G. Baunach, K. O. Netzer, B. Maurer, B. Borisch, and V. ter Meulen. 1990. Infectious DNA of the human spumaretrovirus. Nucleic Acids Res. 18:733-738.

71. Ritter, G. D., Jr., G. Yamshchikov, S. J. Cohen, and M. J. Mulligan. 1996. Human immunodeficiency virus type 2 glycoprotein enhancement of particle budding: role of the cytoplasmic domain. J. Virol. 70:2669-2673.

72. Russell, D. W., and A. D. Miller. 1996. Foamy virus vectors. J. Virol. 70: 217-222.

73. Saib, A., M. H. Koken, P. van der Spek, J. Peries, and H. de The. 1995. Involvement of a spliced and defective human foamy virus in the establishment of chronic infection. J. Virol. 69:5261-5268.

74. Saib, A., M. Neves, M. L. Giron, M. C. Guillemin, J. Valla, J. Peries, and M. Canivet. 1997. Long-term persistent infection of domestic rabbits by the human foamy virus. Virology 228:263-268.

75. Saib, A., J. Peries, and H. Dethe. 1993. A defective human foamy provirus generated by pregenome splicing. EMBO J. 12:4439-4444.

76. Schliephake, A. W., and A. Rethwilm. 1994. Nuclear localization of foamy virus Gag precursor protein. J. Virol. 68:4946-4954.

77. Schmidt, M., O. Herchenroder, J. Heeney, and A. Rethwilm. 1997. Long terminal repeat U3 length polymorphism of human foamy virus. Virology 230:167-178.

78. Schmidt, M., S. Niewiesk, J. Heeney, A. Aguzzi, and A. Rethwilm. 1997. Mouse model to study the replication of primate foamy viruses. J. Gen. Virol. 78:1929-1933.

79. Schweizer, M., R. Renne, and D. Neumann-Haefelin. 1989. Structural analysis of proviral DNA in simian foamy virus (LK-3)-infected cells. Arch. Virol. 109:103-114.

80. Schweizer, M., R. Turek, H. Hahn, A. Schliephake, K. O. Netzer, G. Eder. M. Reinhardt, A. Rethwilm, and D. Neurnann-Haefelin. 1995. Markers of foamy virus infections in monkeys, apes, and accidentally infected humans—appropriate testing fails to confirm suspected foamy virus prevalence in humans. AIDS Res. Hum. Retroviruses 11:161-170.

81. Seeger, C., and W. S. Mason. 1996. Replication of the hepatitis virus genome, p. 815-831. In M. dePamphilis (ed.), DNA replication in eukaryotic cells. Cold Spring Harbor Laboratory Press, Cold Spring Harbor, N.Y.

82. Telesnitsky, A., and S. Goff. 1997. Reverse transcription and the generation of retroviral DNA, p. 121-161. In J. M. Coffin, S. H. Hughes, and H. E. Varmus (ed.), Retroviruses. Cold spring Harbor Laboratory Press, Plainview, N.Y.

83. Trono, D. 1992. Partial reverse transcripts in virions from human immunodeficiency and mutine leukemia viruses. J. Virol 66:4893-4900.

84. Venkatesh, L. K., P. A. Theodorakis, and G. Chinnadural. 1992. Functional dissection of the human spumaretrovirus transactivator identifies distinct classes of dominant-negative mutants. J. Virol. 67:161-169.

85. Wills, J. W., and R. C. Craven. 1991. Form, function, and use of retroviral Gag proteins. AIDS 5:639-654.

86. Winkler, I., J. Bodem, L. Haas, M. Zemba, H. Delius, R. Flower, R. M. Flugel, and M. Lochelt. 1997. Characterization of the genome of feline foamy virus and its proteins shows distinct features different from those of primate spumaviruses. J. Virol. 71:6727-6741.

87. Yang, P., M. Zemba, M. Aboud, R. M. Flugel, and M. Lochelt. 1997. Deletion analysis of both the long terminal repeat and the internal promoters of the human foamy virus. Virus Genes 15:17-23.

88. Yu, S. F., D. N. Baldwin, S. R. Gwynn, S. Yendapalli, and M. I. Linial. 1996. Human foamy virus replication—a pathway distinct from that of retroviruses and hepadnaviruses. Science 271:1579-1582.

89. Yu, S. F., K. Edelmann, R. K. Strong, A. Moebes, A. Rethwilm, and M. L. Linial. 1996. The carboxyl terminus of the human foamy virus Gag protein contains separable nucleic acid binding and nuclear transport domains. J. Virol. 70:8255-8262.

90. Yu, S. F., and M. L. Linial. 1993. Analysis of the role of the bel and bet open reading frames of human foamy virus by using a new quantitative assay. J. Virol. 67:6618-6624.

91. Yu, S. F., J. Stone, and M. L. Linial. 1996. Productive persistent infection of hematopoietic cells by human foamy virus. J. Virol. 70:1250-1254.

92. Yu, S. F., M. D. Sullivan, and M. L. Linial. 1999. Evidence that the human foamy virus genome is DNA. J. Virol. 73:1565-1572.

93. Zemba, M., T. Wilk, T. Rutten, A. Wagner, R. M. Flugel, and M. Lochelt. 1998. The carboxy-terminal p3^{Gag} domain of the human foamy virus Gag precursor is required for efficient virus infectivity. Virology 247:7-13.

94. M. L. Linial, MINIREVIEW, Foamy viruses are unconventional retroviruses, J. Virol. 73 (3) p. 1747-1755 (1999).

TABLE 1

| Feature | Typical Retrovirus | Foamy Retrovirus | Hepadnavirus | Reference |
|---|---|---|---|---|
| Viral polymerase | Reverse | Reverse | Reverse | |
| | transcriptase | transcriptase | transcriptase | |
| Capsid/polymerase | Linked | Independent | Independent | S .F. Yu, D N Baldwin |
| expression | | | | et al 1996, I Jordan et |
| | | | | al, 1996, T Lochelt et |
| | | | | al 1996 |
| Capsid processing | Yes | No | No | reviewed in lineal |
| | | | | 1999 |
| Capsid Nucleic Acid | Cis-His | Gly-Arg | Gly-Arg | S F Yu, K Edelmann, |
| Binding Motif | | | | et al, 1996 |
| Basic DNA binding | No | Yes | Yes | S F Yu, K Edelmann, |
| domain in capsid | | | | et al, 1996 |
| Capsid nuclear | No | Yes | Yes | A W Schliephake and |
| localization | | | | A Rethwilm, 1994 |
| Nucleic acid in | ssRNA | ssRNA, dsDNA | dsDNA | S. F. Yu, D N Baldwin |
| mature virions | | | | et al 1996, S. F Yu, |
| | | | | 1999 |
| Virion Assembly | Plasma (cell) | Endoplasmic | Endoplasmic | T. Kamimura et al., |
| and Budding | | reticulum | reticulum | 1981, P. Roingeard et |
| | | | | al., 1990 |
| Env ER Retention | No | Yes | Yes | P. A. Goepfert et al, |
| “dilysine motif” | | | | 1997 |
| Virion | Envelope | Envelope | Envelope | Bruss and Ganem |
| Assembly/Egress | Independent | dependent | dependent | 1991 |
| Internal Promoters | No | Yes | Yes | T. Lochelt et al, 1993 |
| Transactivation | Yes/No | Yes | Yes | W S Blair, 1994, |
| | | | | Venkatesh et al, |
| | | | | 1992 |
| Intracellular | No | Maybe | Yes | reviewed in lineal |
| Recycling | | | | 1999 |

All references as listed in the paper: MINIREVIEW Foamy Viruses are Unconventional Retroviruses M. L. Lineal, Journal of Virology 73 (3) p 1747-1755 (1999)

What is claimed is:

1. A method for producing a hepadnavirus virion that is infectious in vitro which comprises:
(a) introducing into a cell (i) a hepadnavirus genome expression vector and (ii) a foamy retrovirus envelope expression vector which comprises a nucleic acid encoding at least a fragment of a foamy virus envelope protein; and
(b) culturing the cell thereby producing hepadnavirus virions comprising at least a fragment of a foamy virus envelope protein, wherein the hepadnavirus virions are infectious in vitro:

2. The method of claim 1, wherein the hepadnavirus genome expression vector lacks a nucleic acid encoding a hepadnavirus envelope protein.

3. The method of claim 1, wherein the hepadnavirus genome expression vector comprises at least one gene from a hepadnavirus genome selected from the group consisting of: a wood chuck hepatitis virus (WHV) genome, a ground squirrel hepatitis (GSHV) virus genome, a duck hepatitis B virus (DHBV) genome, a snow goose hepatitis virus (SGHV) genome, and a human hepatitis B virus (HBV) genome.

4. The method of claim 3, wherein the hepadnavirus genome expression vector comprises a gene from a human hepatitis B virus (HBV) genome.

5. The method of claim 3, wherein the hepadnavirus genome expression vector further comprises an exogenous regulatory element.

6. The method of claim 5, wherein the exogenous regulatory element is a human cytomegalovirus immediate-early gene promoter/enhancer (CMV-IE).

7. The method of claim 1, wherein the foamy retrovirus envelope expression vector comprises at least a fragment of a gene from a foamy virus genome selected from the group consisting of: a siman foamy virus (SFV) genome, a feline foamy virus (FFV) genome, a bovine foamy virus (BFV) genome, a sea lion foamy virus (SLFV) genome, a hampster foamy virus (HaFV) genome, and a human foamy virus (HFV) genome.

8. The method of claim 7, wherein the gene encodes an envelope protein or a fragment thereof.

9. The method of claim 1, wherein the foamy retrovirus envelope expression vector comprises a gene or a fragment of a gene from a human foamy virus (HFV) genome.

10. The method of claim 9, wherein the gene or the fragment of the gene from a human foamy virus (HFV) genome encodes the gp130env envelope gene product or a fragment thereof.

11. The method of claim 1, wherein the cell is a mammalian cell.

12. The method of claim 1, wherein the cell is an avian cell.

13. The method of claim 12, wherein the avian cell avian hepacyte.

14. The method of claim 11, wherein the mammalian cell is a human cell.

15. The method of claim 14, wherein the human cell is a human embryonic kidney cell.

16. The method of claim 11, wherein the mammalian cell is a 293 cell.

17. The method of claim 14, wherein the human cell is a human hepatoma cell.

18. The method of claim 17, wherein the human hepatoma cell- is an HepG2 cell or an Huh7 cell.

19. A hepadnavirus virion that is infectious in vitro which comprises at least a fragment of a foamy retrovirus envelope protein.

20. The hepadnavirus virion of claim 19, wherein the hepadnavirus virion is isolated.

21. The hepadnavirus virion of claim 19, wherein the foamy retrovirus is selected from the group consisting of: a siman foamy virus (SFV), a feline foamy virus (FFV), a bovine foamy virus (BFV), a sea lion foamy virus (SLFV), a hampster foamy virus (HaFV), and a human foamy virus (HFV).

22. The hepadnavirus virion of claim 19, wherein the hepadnavirus virion comprises a chimeric envelope protein which consists essentially of (i) a hepatitis B virus envelope protein domain and (ii) a foamy virus envelope protein domain.

23. The hepadnavirus virion of claim 19, wherein the hepadnavirus virion further comprises a nucleic acid isolated from a subject infected by a hepadnavirus.

24. The hepadnavirus virion of claim 23, wherein the nucleic acid isolated from the subject infected by hepadnavirus encodes a reverse transcriptase.

25. The hepadnavirus virion of claim 19, wherein the hepadnavirus further comprises an indicator nucleic acid.

26. A cell comprising the hepadnavirus virion of any one of claims 19 to 25.

27. The cell of claim 26, wherein the cell is a mammalian cell.

28. The cell of claim 27, wherein the mammalian cell is a 293 cell.

29. The cell of claim 27, wherein the mammalian cell is a human cell.

30. The cell of claim 29, wherein the human cell is a human kidney cell.

31. The cell of claim 29, wherein the human cell is a human hepatoma cell.

32. A method for determining susceptibility for an anti- hepadnavirus drug which comprises:
(a) introducing into a first cell:
(i) a hepadnavirus genome expression vector;
(ii) a nucleic acid encoding at least a fragment of a foamy retrovirus envelope protein, and
(iii) an indicator nucleic acid;
(b) culturing the first cell from step (a) so as to produce hepadnavirus virions;
(c) admixing the hepadnavirus virions produced in step (b) with a second cell, wherein the anti-hepadnavirus drug is present with the first cell or the second cell, or with the first and second cell,
(d) measuring the amount of detectable signal produced by the indicator nucleic acid in the second cell, wherein the amount of detectable signal produced is dependent upon hepadnavirus virion infection of the second cell; and
(e) comparing the amount of signal measured in step (d) with the amount signal measured in the absence of the drug, wherein a decrease in the amount of signal measured in the presence of the drug indicates susceptibility to the drug and wherein no change in signal measured or an increase in the amount of signal measured in the presence of the drug indicates resistance to the drug.

33. The method of claim 32, wherein the hepadnavirus genome expression vector of step (a) further comprises a nucleic acid derived from a patient infected with hepadnavirus.

34. The method of claim 33, wherein the nucleic acid derived from a patient infected with hepadnavirus comprises at least a fragment of a human hepatitis B virus (HBV) gene.

35. The method claim 34, wherein the gene is an HBV P gene, an HCV C gene, an HBV X gene or an HBV S gene.

36. The method of claim 34, wherein the nucleic acid derived from a patient infected with hepadnavirus encodes reverse transcriptase.

37. The method of claim 32, wherein the second cell is a mammalian cell

38. The method of claim 32, wherein the second cell is an avian cell.

39. The method of claim 32, wherein the avian cell avian hepacyte.

40. The method of claim 37, wherein the mammalian cell is a human cell.

41. The method of claim 40, wherein the human cell is a human embryonic kidney cell.

42. The method of claim 37, wherein the mammalian cell is a 293 cell.

43. The method of claim 40, wherein the human cell is a human hepatoma cell.

44. The method of claim 43, wherein the hum an hepatoma cell is an HepG2 cell or an Huh7 cell.

45. The method of claim 32, wherein the foamy retrovirus is selected from the group consisting of: a siman foamy virus (SFV), a feline foamy virus (FFV), a bovine foamy virus (BFV), a sea lion foamy virus (SLFV), a hampster foamy virus (HaFV), and a human foamy virus (HFV).

46. The method of claim 32, wherein the nucleic acid of step (a) (i) encodes a gp130env envelope protein.

47. The method of claim 32, wherein the nucleic acid of step (a) (i) encodes a chimeric envelope protein which consists essentially of (i) a hepatitis B virus envelope protein domain and (ii) a foamy virus envelope protein domain.

48. The method of claim 32, wherein the second cell expresses on its surface a protein which binds human foamy virus envelope protein.

49. A method for determining replication capacity of a hepadnavirus from an infected patient comprising:
(a) introducing into a first cell:
(i) a hepadnavirus genome expression vector;
(ii) a nucleic acid encoding at least a fragment of a foamy retrovirus envelope protein, and
(iii) an indicator nucleic acid;
(b) culturing the cell from (a) so as to produce hepadnavirus virions;
(c) admixing the hepadnavirus virions produced in step (b) with a second cell, (d) measuring the amount of detectable signal produced by the indicator nucleic acid in the second cell, wherein the amount of detectable signal produced is dependent upon hepadnavirus virion infection of the second cell;
(e) normalizing the measurement of step (d); and
(f) comparing the normalized measurement of step (e) with the amount signal measured when steps (a) through (d) are carried out with a control reference hepadnavirus, wherein an increase in signal compared to the control indicates an increased replication capacity and a decrease in signal measured compared to the control indicates a decreased replication capacity of the hepadnavirus from the infected patient.

50. A method for determining susceptibility for an anti-hepadnavirus drug which comprises:
(a) introducing into a cell:
(i) a hepadnavirus genome expression vector;
(ii) a nucleic acid encoding at least a fragment of a foamy retrovirus envelope protein, and
(iii) an indicator nucleic acid;
(b) culturing the cell from step (a);
(c) contacting the cell with the anti-hepadnavirus drug;
(d) measuring the amount of detectable signal produced by the indicator nucleic acid in the cell; and
(e) comparing the amount of signal measured in step (d) with the amount signal measured in the absence of the drug, wherein a decrease in the amount of signal measured in the presence of the drug indicates susceptibility to the drug and wherein no change in signal measured or an increase in the amount of signal measured in the presence of the drug indicates resistance to the drug.

51. The method of claim 50, wherein wherein the hepadnavirus genome expression vector of step (a) further comprises a nucleic acid derived from a patient infected with hepadnavirus.

52. The method of claim 51, wherein the nucleic acid derived from a patient infected with hepadnavirus comprises at least a fragment of a human hepatitis B virus (HBV) gene.

53. The method of claim 52, wherein the gene is an HBV P gene or an HBV C gene.

54. A method for identifying a mutation in a hepadnavirus nucleic acid that confers resistance to an anti-hepadnavirus drug which comprises:
(a) sequencing the hepadnavirus nucleic acid prior to use of the anti-hepadnavirus drug;
(b) measuring susceptibility of the hepadnavirus sequenced in step (a) to the drug according to the method of claim 50;
(c) exposing the hepadnavirus to the drug so as to produce a decrease in the susceptibility of the hepadnavirus to the drug measured in step (b);
(d) comparing the sequence determined in step (a) with the sequence of the hepadnavirus following the exposure to the drug of step (c) so as to identify a mutation in the hepadnavirus nucleic acid that confers resistance to the anti-hepadnavirus drug.

55. The method of claim 54, wherein measuring step (b) comprises measuring susceptibility of the hepadnavirus sequenced in step (a) to the anti-hepadnavirus drug according to the method of claim 32.
